(19) United States
(12) Patent Application Publication
(10) Pub. No.: US 2003/0232389 A1
(43) Pub. Date: Dec. 18, 2003

(54) Urokinase peptide structure mimetics

(76) Inventors: Olaf G Wilhelm, (US); Markus Bürgle, M?uuml;nchen (DE); Horst Kessler, Schwalbach (DE); Niko Schmiedeberg, Rhein (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/362,184

(22) PCT Filed: Aug. 21, 2001
(86) PCT No.: PCT/EP01/09668

(30) Foreign Application Priority Data
Aug. 23, 2000 (EP) 00118099.1

(51) Int. Cl. G01N 33/53; A61K 38/17; G06F 19/00; G01N 33/48; G01N 33/50

(52) U.S. Cl. 435/7.1; 514/12; 702/19

(57) ABSTRACT

The NMR structure of the peptidic urokinase type plasminogen activator antagonist cyclo[21,29][D-Cys21Cys29]-uPA₂₁₋₃₀ has been solved to identify design strategies for peptidomimetics that interfere with the binding of urokinase type plasminogen activator with its receptor.

The present invention concerns the use of the NMR structure of cy-clo[21,29][D-Cys21Cys29]-uPA₂₁₋₃₀for the design of inhibitors that interfere with the binding of urokinase to its receptor, and it concerns peptidomimetics that imitate the binding mode of cyclo[21,29][D-Cys21Cys29]-uPA₂₁₋₃₀ to its receptor and therefore interfere with the binding of urokinase to its receptor.

Urokinase-type plasminogen activator (uPA) is a serine protease that is secreted as a single chain proenzyme. Limited proteolysis leads to the generation of the mature, two chain form of the enzyme, that catalyzes the conversion of the zymogen plasminogen to plasmin. Plasmin directs the degradation of the extracellular matrix either directly or indirectly via the activation of matrix metalloproteinases. Therefore, uPA plays a major role in matrix degradation, both in physiological and pathophysiological processes. In metastasis, uPA is an important factor, because it helps tumors to invade the surrounding tissue.

uPAR (uPA receptor) is a glycosyl-phosphatidylinositol (GPI) linked cell surface protein, that binds uPA with subnanomolar affinity. It recruits uPA to the cell surface. The importance of the uPA binding to uPAR for tumor spread has been demonstrated in many cases. Conversely, the addition of a recombinant solubable form of the receptor reduced the invasive capacity of ovarian cancer cells (Wilhelm et al., FEBS Lett. 337 (1994), 131-134).

As a result, uPA antagonists that block the interaction of uPA with its receptor can be used for the treatment of invasive tumors. Other indications for uPA antagonists include conditions such as arthritis, inflammation and osteoporosis. uPA antagonists can also be used as contraceptives.

A successful strategy to design uPA antagonists has built on the modular organisation of uPA. The molecule consists of (a) a growth factor domain (GFD, amino acids 1-44 and 46, respectively), (b) a kringle domain (amino acids 45 and 47, respectively, to 135), that together form the amino terminal fragment (ATF), and (c) a serine protease domain. It was found that ATF, and in particular residues 20-30 of the so-called loop B of GFD, compete efficiently with uPA for binding to uPAR.

Wilhelm et al. have investigated cyclic disulfide peptides that mimick this loop. Their studies identified cyclo[21,29][D-Cys21Cys29]-uPA₂₁₋₃₀ with an IC50 of 78 nM as a particularly promising drug lead (German patent application 199 33 701.2). Residues in this cyclic peptide cyclo[1,9] D-Cys-Asn-Lys-Tyr-Phe-Ser-Asn-Ile-Cys-Trp will be numbered sequentially, assigning residue number 1 to D-Cys. Thus, residue 1,2,3 . . . of the cyclic peptide correspond to residues 21,22,23, . . . in the ATF of uPA.

Although replacement of the Lys residue abolishes the susceptibility of the Lys-Tyr bond to the proteolytic action of plasmin (German patent application 199 33 701.2), it is expected that the peptide still suffers from some of the disadvantages of peptide drugs. These include lability against proteolysis in the stomach/intestine, low resorption if administered perorally, fast elimination by the liver and kidney and the risk of allergic reactions. Due to their conformational flexibility, peptide drugs and/or their metabolic products may interact with molecules other than their target molecules, leading to side effects that are both unwanted and hard to predict.

It is therefore an object of the present invention to provide inhibitor molecules that do not suffer from the above-mentioned disadvantages of the peptide lead compound and still maintain the affinity for uPAR.

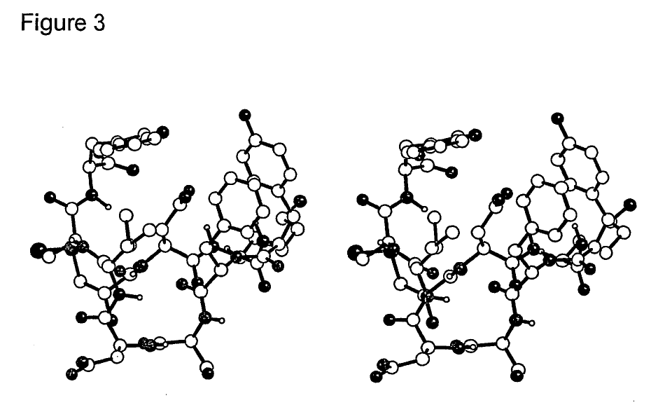

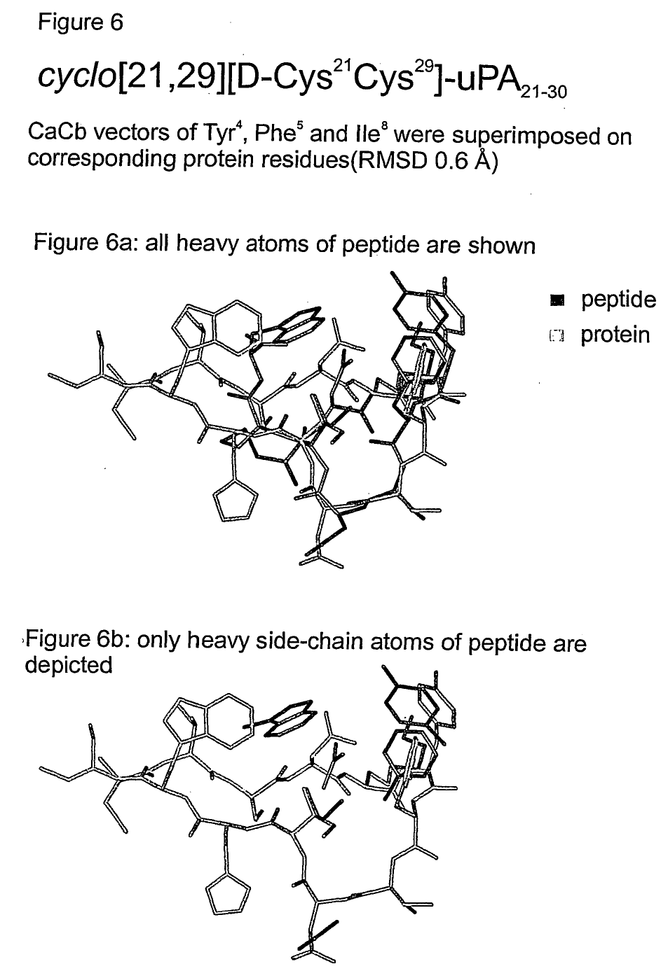

This object is solved with the determination of the NMR solution structure of the lead compound, cyclo[21,29][D-Cys21Cys29]-uPA_{21-30.} The procedure for structure determination is described in detail in Example 1 and the result is presented as a stereo representation of the molecule in FIG. 3 and as a coordinate file in FIG. 6.

It is a further object of the present investigations to provide molecules that mimick the lead compound cyclo[21,29][D-Cys21Cys29]-uPA₂₁₋₃₀.

In an embodiment of the invention, conformation stabilizing cycles such as

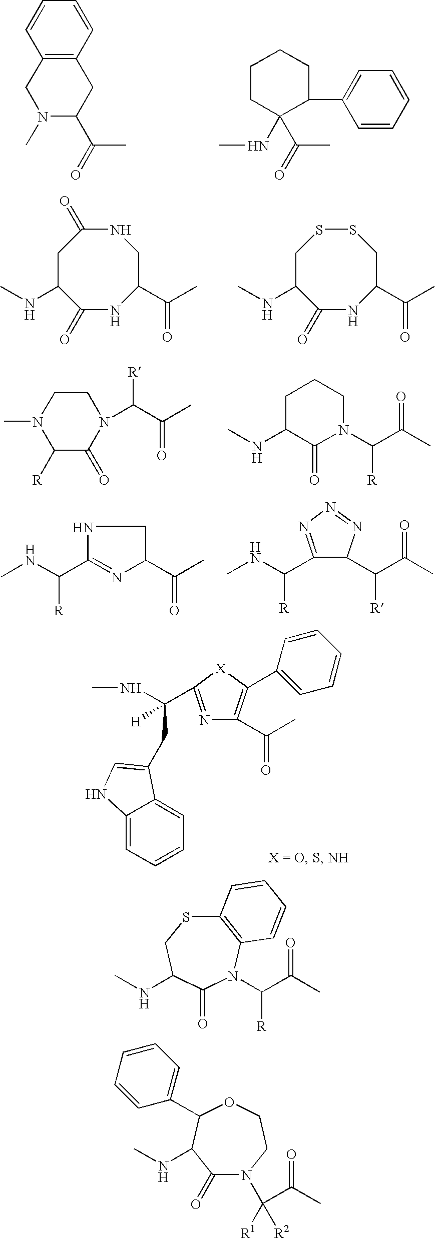

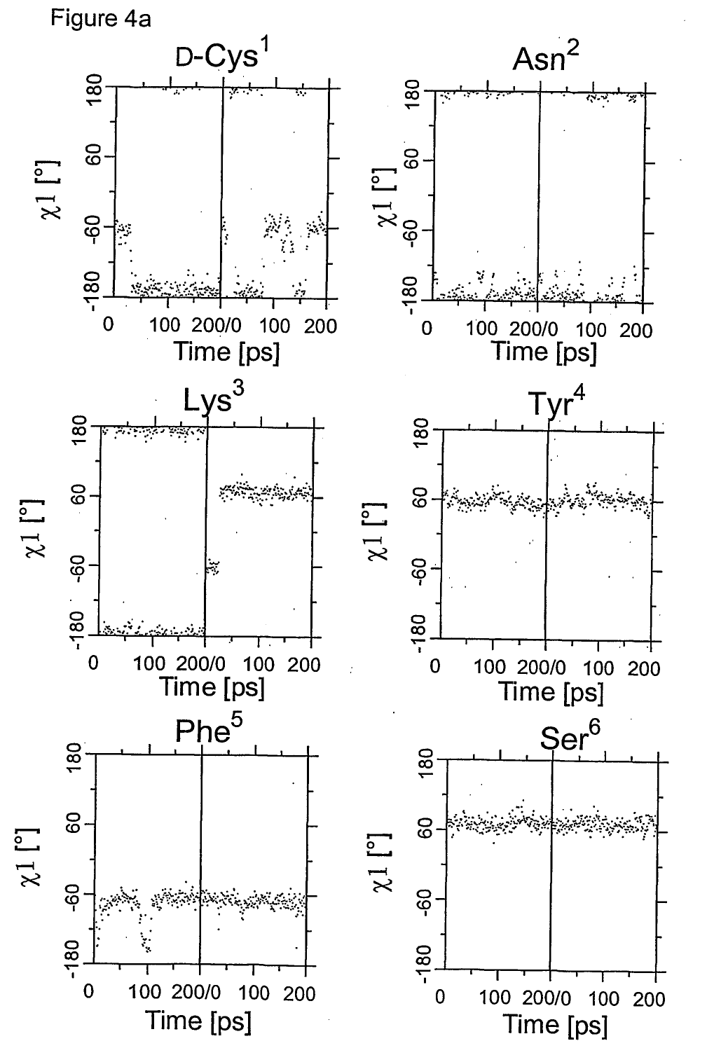

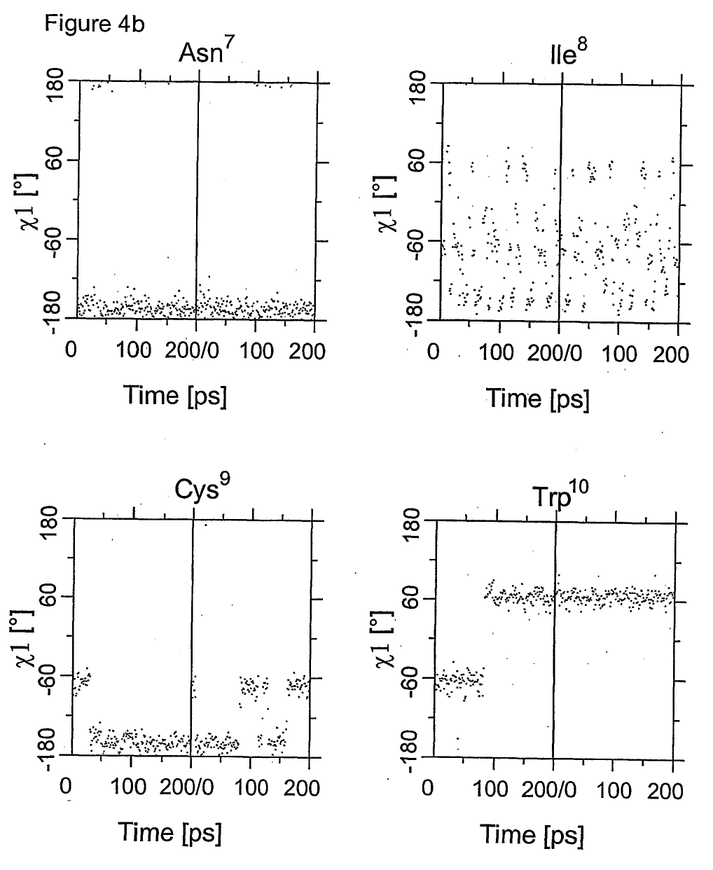

are chosen for incorporation into the peptide, so that Ramachandran angles actually found in the lead peptide are enforced by the additional cycles (Gante, Angew.Chemie 1994, 106:1780-1802). In another preferred embodiment, conformationally constrained amino acid analogs are used to limit space (Gibson, S. E., Guillo, N., Toser, M. J., Tetrahedron 1999, 55:585-615) to regions actually used by the cyclic peptide and identified as part of this invention (see FIG. 4).

In another embodiment of the invention, β-turn mimetics

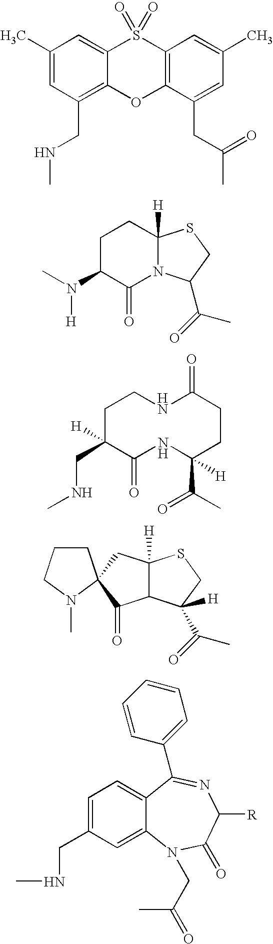

(Gante, J., Angew.Chemie 1994, 106:1780-1802; Böhm, H. J., Klebe, G., Kubinyi H., Wirkstoffdesign, Spektrum Adamischer Verlage, Gannis, A., Kolter, T., Angew. Chemie 1993, 105:1303-1326) are chosen to replace the type Iβ-turn forming tetrapeptides Asn-Lys-Tyr-Phe and/or Phe-Ser-Asn-Ile.

In a preferred embodiment β-turn mimetics that allow the attachment of side chains in positions i+1 and i+2 are used. Such scaffolds are for example the β-D-glucose scaffold (Nicolaou et al., Pept. Chem. Struct. Biol. Proc. Am. Pept. Symp. 11th, 1989 (1990), 881) or the cyclohexane scaffold (Olson et al., Proc. Biotechnol (USA), Conference Management Corporation, Norwalk, Conn., 1989, p.348).

In another embodiment of the invention, two subsequent residues with Rarnachandran angles typical of residues in an α-helical arrangement are replaced with α-helix inducing mimetics such as

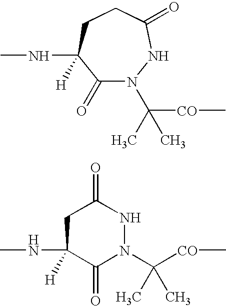

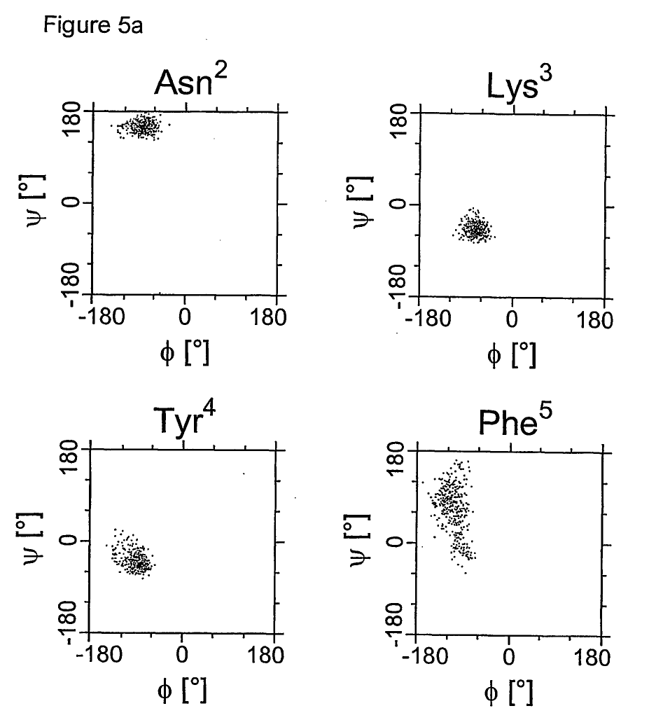

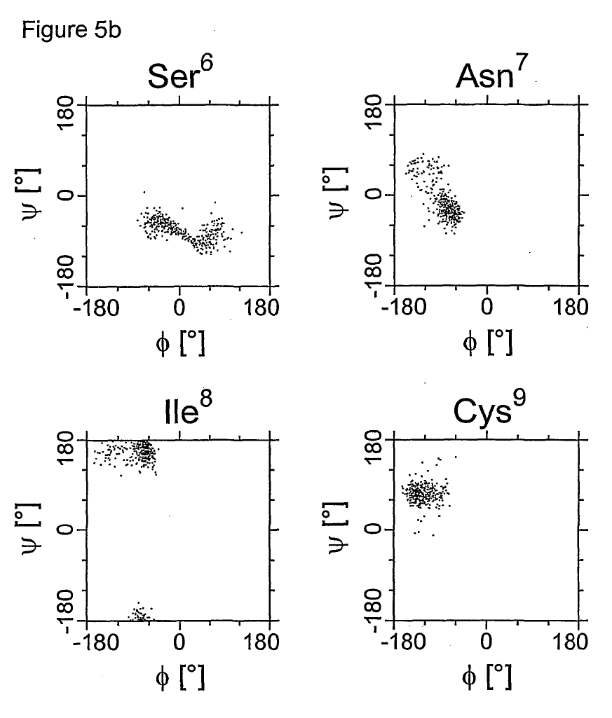

As shown in FIG. 5, such subsequent residues in cyclo[21,29][D-Cys21Cys-29]-uPA₂₁₋₃₀ are Lys3/Tyr4 and/or Ser6/Asn7.

In another embodiment of the invention, the polypeptide backbone is altered in such a way that the orientation of side chains is not substantially altered. Modifications include replacement of a peptide amido group with a ketomethylene, hydroxyethylene or ethylene group, leading to the formation of carbapeptide moieties in the molecule. The converse strategy, replacement of an α-carbon with a substituted nitrogen atom is equally possible and leads to the formation of azapeptide moieties. Azapeptides can be formed conviniently by condensing carboxyterminally acitivated azaamino acids.

In another embodiment of the invention, the two strategies of the preceding paragraphs are combined to form peptoid (Simon et al., Proc. Nat. Acad. Sci. USA 89, 9367 (1992) moieties. Peptoids contain nitrogen atoms instead Cα-atoms and carbon atoms instead of the α-amino nitrogen atoms, such that an NR—CO peptide-like bonded chain of N-alkylated glycines is formed.

The present invention additionaly concerns a pharmaceutical composition which contains at least one peptide or polypeptide or analogue thereof as defined above as the active substance, optionally together with common pharmaceutical carriers, auxilliary agents or diluents. The peptides or polypeptides according to the invention are used especially to produce uPA antagonists which are suitable for treating diseases associated with the expression of uPAR and especially for treating tumors.

An additional subject matter of the present invention is the use of peptides derived from the uPA sequence and in particular of uPA antagonists such as the above mentioned peptides and polypeptides to produce targeting vehicles e.g. liposomes, viral vectors etc. for uPAR-expressing cells. The targeting can be used for diagnostic applications to steer the transport of marker groups e.g. radioactive or non-radioactive marker groups. On the other hand, the targeting can be for therapeutic applications e.g. to transport pharmaceutical agents and for example also to transport nucleic acids for gene therapy.

The pharmaceutical compositions according to the invention can be present in any form, for example as tablets, as coated tablets or in the form of solutions or suspensions in aqueous and non-aqueous solvents. The peptides are preferably administered orally or parenterally in a liquid or solid form. When they are administered in a liquid form, water is preferably used as the carrier medium which optionally contains stabilizers, solubilizers and/or buffers that are usually used for injection solutions. Such additives are for example tartrate of borate buffer, ethanol, dimethyl sulfoxide, complexing agents such as EDTA, polymers such as liquid polyethylene oxide etc.

If they are administered in a solid form, then solid carrier substances can be used such as starch, lactose, mannitol, methyl cellulose, talcum, highly dispersed silicon dioxide, high molecular weight fatty acids such as stearic acid, gelatin, agar, calcium phosphate, magnesium stearate, animal and vegetable fats or solid high molecular polymers such as polyethylene glycols. The formulations can also contain flavourings and sweeteners if desired for oral administration.

The therapeutic compositions according to the invention can also be present in the form of complexes e.g. with cyclodextrins such as γ-cyclodextrin.

The administered dose depends on the age, state of health and weight of the patient, on the type and severity of the disease, on the type of the treatment, the frequency of administration and the type of desired effect. The daily dose of the active compound is usually 0.1 to 50 mg/kilogramme body weight. Normally 0.5 to 40 and preferably 1.0 to 20 mg/kg/day in one or several doses are adequate to achieve the desired effects.

### EXAMPLE 1

Abbreviations: SA, simulated annealing; MD, molecular dynamics; rMD, restrained molecular dynamics; fMD, free molecular dynamics; NOE, nuclear Overhauser enhancement; RMSD, root mean square deviation; uPA, urokinase-type plasminogen activator; ATF, amino-terminal fragment of uPA;

Materials and Methods

NMR Spectroscopy. All NMR spectra were acquired on a Bruker DMX600 spectrometer and processed using the X-WINNMR software. A set of 1D spectra was acquired at the following temperatures: 275 K, 276 K, 278 K, 280 K, 282 K, 284 K and 285 K. COSY and NOESY spectra were acquired in water with 1024 and 512 complex points in t2 and t1, respectively, performing 64 scans per increment. A mixing time of 80 ms was chosen for the NOESY. Water suppression was accomplished using WATERGATE. The E.COSY spectrum was recorded in D₂O at a resolution of 4096(t2)*256(t1) complex points, with 48 scans per increment. All 2D spectra were recorded at 280 K.

NOE-Derived Distance Restraints. NOE crosspeaks were converted into distance restraints d_{NOE} according to their integrated volumes using the two-spin approximation. The lower and upper bound of each distance restraint was set to 0.9 d_{NOE} and 1.1 d_{NOE}, respectively. The average intensity of NOEs between geminal methylen protons (corresponding to a distance of 1.8 Å) was used for calibration, Standard corrections for center averaging [1] were applied.

Coupling Constants. ³J(H^{N}H^{α}) were obtained from the COSY spectrum using the methodology pioneered by Kim and Prestegard ^{[2]}. ³J(H^{α}H^{β}) were extracted from the E.COSY recorded in D₂O.

Amide Proton Temperature Coefficients. Temperature dependencies of the backbone amide proton chemical shifts were calculated from the above temperature series of ¹H-1D experiments.

Structure Calculations. Structure calculations consisted of a two-step procedure involving conformational space sampling followed by refinement of the obtained three-dimensional structure. In vacuo conformational space sampling was performed with the X-PLOR 3.5 program^{[3]} employing a standard simulated annealing (SA) protocol. ^{[4,5]} A random conformation with optimized covalent bond geometries was used as the initial structure for all calculations. NOE-derived distances as well as ³J(H^{N}H^{α}) coupling constants were employed as restraints. Ten low-energy conformations out of a total of 20 generated structures were selected for analysis of the agreement with the NMR-derived restraints. A structural representative of the ensemble of low-energy structures was then chosen and refined in extensive molecular dynamics (MD) simulations. To this end, the representative was placed in a 35 Å cubic simulation cell soaked with water molecules. The simulation cell was then energy-minimized and slowly heated up to the target temperature of 280 K. After equilibration, 200 ps restrained MD (rMD) were performed. Solely NOE-derived distances were employed, acting as time averaged distance restraints ^{[6-9]} with a memory decay time of r=20 ps. ^{[9]} To obtain average properties, the above simulation protocol was carried out twice, starting from different initial velocities. Finally, one MD simulation was resumed in absence of restraints to probe the stability of the structure (free MD, fMD). All MD simulations were performed with the DISCOVER 98 program (Molecular Simulations Inc.) using a home-written C program handling the time averaging of distance restraints.

Results and Discussion

Nomenclature. For sake of clarity residues of cyclo[21,29][D-Cys²¹,Cys²⁹] uPA₂₁₋₃₀ will be numbered from 1 through 10 in the following, while for the corresponding residues of the ATF of uPA the original numbering scheme is retained.

NMR Assignments. The ¹H chemical shifts (Table 1) were assigned from analysis of the COSY and NOESY spectra. In the first step of the assignment procedure, frequencies of non-aromatic protons of each of the amino acid spin systems were determined using the COSY spectrum. Next, frequencies of aromatic protons were obtained from the NOESY spectrum. To this end, the chain of strong NOEs between adjacent protons in each aromatic side chain was traced, starting from the H^{β} protons. Finally, the sequential order of the amino acid spin systems was determined using characteristic H^{α}ᵢ-H^{N}ᵢ₊₁-NOEs as well as interresidue side-chain NOEs. A comparison of the obtained ¹H chemical shifts with the corresponding random coil values (Wüthrich, K., NMR of Proteins and Nucleic Acids, Wiley, N.Y., 1996) reveals a considerable upfield shift for Lys³ (random coil chemical shifts are given in parentheses; H^{β}: 1.33, 1.45 (1.76, 1.85); H^{γ}: 0.54, 0.79 (1.45); H^{δ}: 1.24 (1.70)) and Ile⁸ (γCH₃: 0.42 (0.95), δCH₃: 0.48 (0.89)) side-chain protons, which is due to aromatic ring systems adjacent in space (see Structure section).

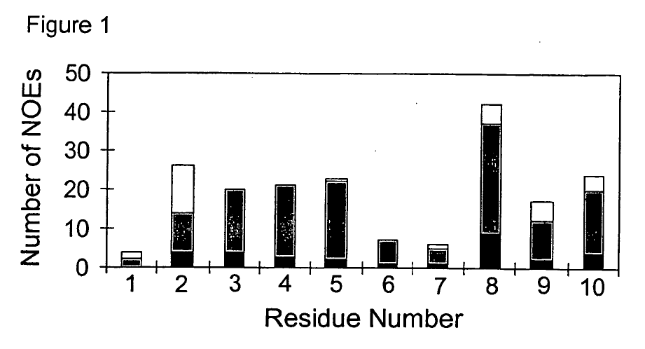

NMR-Derived Structure Parameters. A total of 110 unambiguous NOE-derived distance restraints was obtained from analysis of the NOESY spectrum, including 30 nontrivial intraresidue, 40 sequential, 25 short-range (|i−j|<5, where i and j are residue numbers), and 15 long-range (|i−j|≧5) NOEs. Due to signal overlap in the 2D NOESY spectrum, a considerable amount of structural information is lost (see similarity of chemical shift values given in Table 1). A histogram of the NOE restraints for each residue is shown in FIG. 1. Aside from NOE-derived distances, nine ³J(H^{N}H^{α}) (Table 2) and an almost complete set of ³J(H^{α}H^{β}) (Table 3) coupling constants were obtained from analysis of the COSY and E.COSY spectra. NOESY signal overlap and/or averaged ³J(H^{α}H^{β}) coupling constants due to side-chain rotation (Table 3) did not allow for diastereotopic assignment of H^{β}. In addition to NOE distances and vicinal coupling constants, temperature dependencies of the chemical shifts from six out of a total of nine backbone amide protons were obtained from the temperature series of 1D spectra.

Conformational Space Sampling. Only one family of backbone conformations was observed during conformational space sampling in vacuo using X-Plor (average backbone RMSD 0.6 Å from the family representative for residues 2 through 8). As already mentioned in the above paragraph, a considerable amount of signals in the 2D NOESY spectrum overlap, giving rise to ambiguous distance restraints. However, ambiguous distance restraints cannot be treated in the current version of the DISCOVER program which is used for subsequent refinement. To probe whether the set of ambiguous distance restraints influences the convergence of the X-Plor runs, three-dimensional structures were generated with and without incorporation of ambiguous distance restraints. The results are virtually identical (backbone RMSD between structural representatives 0.5 Å for residues 2 through 8). Thus, the set of unambiguous distance restraints already contains the principal structural information. Therefore, only unambiguous distance restraints were employed in the refinement stage.

Structural Refinement. The single structural representative obtained during conformational space sampling was refined in the course of 200 ps rMD simulations. To obtain average properties, two simulations were performed, starting from the same system configuration but different initial velocities. Both rMD simulations lead to similar results (backbone RMSD between energy-minimized average structures 0.3 Å for residues 2 through 8). To probe the stability of the rMD structure, one simulation was resumed in absence of restraints for another 200 ps (fMD). An inspection of the Ramachandran plots of the fMD trajectory (not shown) reveals that the rMD conformation is retained, a finding which is confirmed by the backbone RMSD between the energy-minimized average structures of both simulations (0.9 Å for residues 2 through 8).

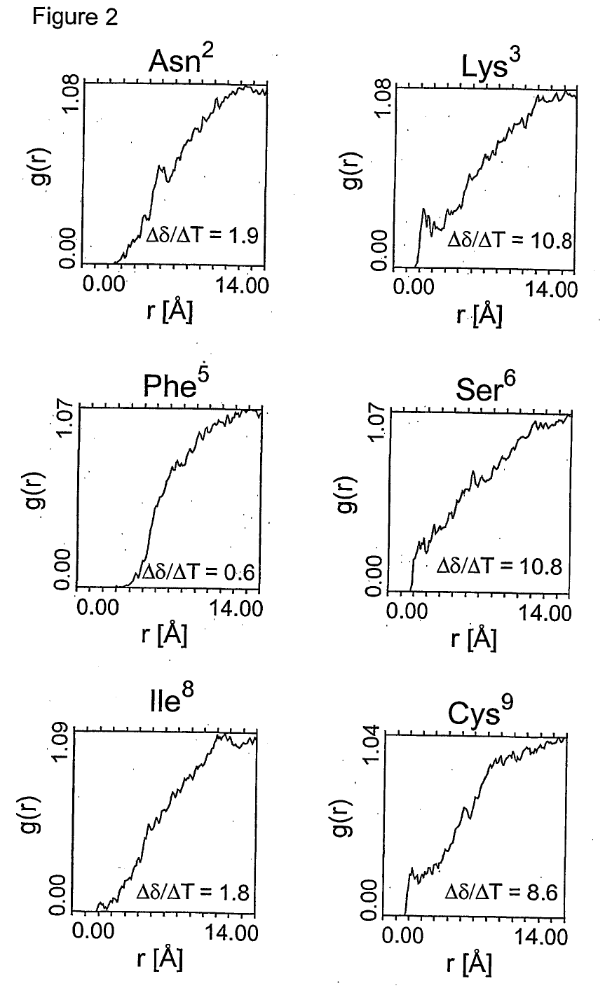

According to analysis of the joint rMD trajectories (in the following denoted as rMD trajectory), the average violation of NOE-derived distance restraints is 0.1 Å with no single distance restraint violated by more than 0.5 Å. Although coupling constants were not employed as restraints in the refinement stage, ³J(H^{N}H^{α}) calculated from the rMD trajectory are close to their experimental values (Table 2). Deviations by more than 2 Hz can be explained in terms of the steep gradient of the corresponding Karplus curve at φ=−80±30° (curve not shown). Similar considerations apply for ³J(H^{α}H^{β}). Despite the fact that no diastereotopic assignment of H^{β} was possible, a comparison of calculated versus experimental values of ³J(H^{α}H^{β}) yields similar pairings (Table 3), suggesting that the side-chain rotamer distribution is correctly reproduced by the rMD trajectory. Deviations occur for Tyr⁴, Ser⁶, Asn⁷ and Trp¹⁰. In case of Ser⁶, no NOE-derived distance restraints are available due to signal overlap. Therefore, the calculated rotamer distribution merely reflects the force-field preferences. This is also true for Asn⁷, where NOEs to the H^{β} are present, but, due to the fact that the DISCOVER program cannot handle pseudo atoms under periodic boundary conditions, act on the C^{β} atom, thereby eliminating their influence on the X¹ rotamer distribution. Deviations of the experimental ³J(H^{α}H^{β}) values of Tyr⁴ and Trp¹⁰ will be discussed in conjunction with the three-dimensional structure of the molecule (see Section Structure and Dynamics). Temperature dependancies of backbone amide proton chemical shifts are in good agreement with the corresponding amide proton solvent accessibilities calculated from the rMD trajectory (FIG. 2).

Structure and Dynamics of cyclo[21,29][D-Cys²¹,Cys²⁹]uPA₂₁₋₃₀. The three-dimensional structure of the molecule is characterized by a hydrophobic cluster on one side of the ring, involving residues Tyr⁴, Phe⁵, Ile⁸ and Trp¹⁰, and two type βI turns centered at Lys³, Tyr⁴ and Ser⁶, Asn⁷, respectively (FIG. 3).

All hydrophobic residues (Tyr⁴, Phe⁵, Ile⁸ and Trp¹⁰) participate in the formation of a hydrophobic cluster. Ile⁸ is found at the core of the cluster, with its side chain being shielded from the aqueous environment by the phenyl ring of Phe⁵ and the indole moiety of Trp¹⁰. This finding is consistent with the distinct upfield shift observed for the chemical shifts of the methyl groups of the isoleucine side chain, suggesting these methyls to be located above the plane of aromatic ring systems (see section NMR Assignment). However, the nature of the hydrophobic cluster is not as static as FIG. 3 might suggest. As can be seen in FIG. 4, Ile⁸ displays remarkable flexibility around X¹. According to one larger and one smaller ³J(H^{α}H^{β}) value (Table 3), Tyr⁴ partially adopts the g⁻ and t rotamer, while in the rMD simulation only the g⁻ rotamer is populated (FIG. 4), allowing for the formation of a hydrophobic cluster with Phe⁵ (FIG. 3). In contrast, the g⁻ rotamer enables a hydrophobic interaction with the methylens of the lysine side chain, a feature also found in the corresponding ω loop in the NMR solution structure of the ATF of uPA.^{[10]} The resulting spatial arrangement would still be consistent with the observed NOEs between the side chains of Lys³ and Tyr⁴ and could also account for the distinct upfield shift of the β, γ and δ protons of the lysine side chain (see section NMR Assignment). In case of Trp¹⁰, the experimental evidence (both ³J(H^{α}H^{β}) around 7.0 Hz, upper bound of H^{α}-H² distance restraint violated) also indicates side-chain rotation, albeit not reproduced in the rMD simulation (FIG. 4). Rotation around X¹ would bring the indole ring of Trp¹⁰ in a position comparable to that observed for its counterpart in the solution structure of the ATF. Obviously, the chosen time averaging regime for NOE-derived distance restraints using a memory decay time T of 20 ps^{[9]} does not allow for side-chain rotational fluctuations large enough to correctly reproduce the experimental ³J(H^{α}H^{β}) values.

In addition to a hydrophobic cluster, the molecule also displays regular secondary structure. A type βI turn (ideal φ,ψ dihedral values: −60°, −30° (i+1 position) and −90°, 0° (i+2 position))^{[11,12]} is centered at Lys³ and Tyr⁴ (FIG. 5, FIG. 3). The corresponding (i,i+3) hydrogen bond is not populated to an appreciable extent, a phenomenon also encountered in 25% of the β-turns found in protein structures.^{[13]} The turn is stabilized by a sidechain-backbone hydrogen bond between Asn²O^{δ1} and the amide proton of Tyr⁴, forming another turn-like structure known as “Asx turn”.^{[14]} In addition, Asn²O^{δ1} hydrogen-bonds to Phe⁵H^{N}, providing a rationale for the weakly populated (i,i+3) hydrogen bond of this βI turn (Table 4). Another type βI turn is centered at Ser⁶ and Asn⁷, with the corresponding (i,i+3) hydrogen bond between Phe⁵CO and Ile⁸H^{N} populated in more than half of the rMD simulation time (Table 4). An equally populated hydrogen bond between Ser⁶O^{γ} and Asn⁷H^{N} stabilizes the ψᵢ₊₁ angle of this turn (Table 4). In the course of the rMD simulation, the Phe5-Ser⁶ amide bond rotates (FIG. 5), giving rise to a weakly populated type γ turn centered at Ser⁶ (Table 4) with the φᵢ₊₁ angle stabilized by an additional sidechain-backbone hydrogen bond between Phe⁵CO and Ser⁶H^{γ} (Table 4). The φ,ψ pairs of this turn are close to their ideal values (70°, −70°).^{[11,12]} The observed arrangement of two consecutive type βI turns is additionally stabilized by a strongly populated hydrogen bond between Asn²H^{N} and Ile⁸CO (Table 4).

Agreement with statistically determined β-turn positional preferences. The large body of experimental information on the three-dimensional structure of proteins available in the Brookhaven Protein Data Bank^{[15]} has enabled conformational and positional preferences of residues to be statistically determined.^{[16-20]} Using a nonhomologous dataset of 205 protein chains, Hutchinson and Thornton derived β-turn positional potentials for the 20 naturally occuring amino acids.^{[20]} For position i of type βI turns, they found a strong preference for side chains that can act as hydrogen bond acceptors (Asn, Asp, Cys, Ser, His). These stabilize the turn by the formation of a hydrogen bond with the main-chain nitrogen of the i+2 residue. Thereby another turn-like structure known as “Asx turn” ^{[14]} arises, made up of the side chain and main chain of residue i, together with the main chains of residues i+1 and i+2. For the remaining positions of type βI turns, Hutchinson and Thornton found significant positional preferences for the following residues: i+1: Pro, Ser, Glu; i+2: Thr, Ser, Asn, Asp; i+3: Gly. Indeed, an “Asx turn” is observed for the type βI turn centered at Lys³ and Tyr⁴ of cyclo[21,29][D-Cys²¹,Cys²⁹]uPA₂₁₋₃₀, bearing Asn² in position i (see section Structure and Dynamics). However, none of the other residues of this βI turn (Lys³ in i+1, Tyr⁴ in i+2, and Phe⁵ in i+3 position) displays significant propensity to appear in its respective position. In contrast, Ser⁶ and Asn⁷ in i+1 and i+2 position, respectively, of the second βI turn are in perfect agreement with the statistically derived preferences (see above). Ser⁶O^{γ} hydrogen-bonds to Asn⁷H^{N}, thereby stabilizing the ψⁱ⁺¹ angle. As for position i+2, an analysis of high-resolution protein structures shows that Asn, along with Asp, Ser and Thr, is more likely to adopt the backbone conformation required for this position (φ=−90°, ψ=0°).^{[21]}

Comparison with Solution Structure of Amino-Terminal Fragment of uPA. Cyclo[21,29][D-Cys²¹,Cys²⁹]uPA₂₁₋₃₀ and the ATF of uPA display similar binding characteristics with respect to the uPA receptor (uPAR). Thus, similar orientations of residues critical for receptor binding can be expected. These residues comprise Tyr²⁴, Phe²⁵, Ile²⁸, and Trp³⁰ within the ω loop of ATF [22] and the corresponding residues Tyr⁴, Phe⁵, Ile⁸, and Trp¹⁰ in our cyclic peptide, as determined by alanine replacements. Superposition with the solution structure of ATF ^{[10]} reveals that residues Tyr²⁴ (Tyr⁴ in the cyclic peptide), Phe²⁵ (Phe⁵), and Ile²⁸ (Ile⁸) indeed adopt indentical positions and orientations relativ to each other (RMSD between C^{α}-C^{β} vectors of corresponding tyrosine, phenylalanine and isoleucine residues 0.6 Å, see also FIG. 6). Trp³⁰ (Trp¹⁰), however, is found in different orientations in both uPAR ligands. In the cyclic peptide, Trp¹⁰ is located outside the cyclic backbone of the peptide, which confers considerable conformational flexibility to this C-terminal residue. Thus, Trp¹⁰ can participate in the formation of the observed hydrophobic cluster, together with Tyr⁴, Phe⁵ and Ile⁸. Upon receptor binding, however, its conformational flexibility enables Trp¹⁰ to bring its indole in a position comparable to that found in the ATF. Interestingly, the presence of Phe and Trp seperated by five residues in sequence is among the essential features of uPAR binding peptide antagonists identified by phage display technology.^{[23]} The consensus sequence derived from these linear peptides is XFXXYLW. The importance of proper spacing is further corroborated by the experimental finding that insertion of either Gly or β-Ala between Phe and Trp results in loss of antagonist function.^{[24]} Furthermore, a manual alignment of our peptide and the above consensus sequence reveals the hydrophobic residues Ile⁸ and the consensus Tyr to be located in equivalent positions. Thus, formation of a hydrophobic cluster between Phe and Ile (Tyr), as observed for our peptide, as well as an appropriately spaced Trp seem to constitute preconditions for high affinity binding to uPAR.

Besides the above hydrophobic residues, substitution of Ser⁶ by Ala also results in weaker binding to uPAR. This observation can be explained in terms of the structure-stabilizing effect of the serine residue by sidechain-backbone hydrogen bonds, as described in section Structure and Dynamics.

TABLE 1
¹H chemical shifts [ppm] of cyclo[21,29][D-Cys²¹,Cys²⁹]-uPA₂₁₋₃₀ in water at 280 K.^{a}

| Residue | H^{N} | H^{α} | H^{β} | H^{γ} | H^{δ} | H^{ε} | misc. | |
|---|---|---|---|---|---|---|---|---|
| D-Cys¹ | — | 3.81 | 2.62/3.26 | — | — | — | — | |
| Asn² | 8.57 | 4.51 | 2.62/2.79 | — | 6.99/7.38 | — | — | |
| Lys³ | 8.74 | 3.73 | 1.33/1.45 | 0.54/0.79 | 1.24 | 2.57 | 7.32 | (HN^{ε}) |

| | | | | | | | | |
|---|---|---|---|---|---|---|---|---|
| Tyr⁴ | 8.03 | 4.16 | 2.52/2.62 | — | — | — | 6.86 | (H^{2,6}) |
| | | | | | | | 6.57 | (H^{3,5}) |
| Phe⁵ | 7.59 | 4.61 | 2.46/3.12 | — | — | — | 6.99 | (H^{2,6}) |
| | | | | | | | 7.06 | (H^{3,5}) |

| Ser⁶ | 8.40 | 3.91 | 3.70/3.79 | — | | — | — | — |
|---|---|---|---|---|---|---|---|---|
| Asn⁷ | 8.00 | 3.97 | 2.34/2.86 | — | | 6.79/7.48 | — | — |
| Ile⁸ | 7.42 | 3.76 | 1.56 | 0.91/1.16 | (CH₂) | 0.48 | | |
| | | | | 0.42 | (CH₃) | | | |

| Cys⁹ | 8.31 | 4.58 | 2.74/3.01 | — | — | — | — | |
|---|---|---|---|---|---|---|---|---|
| Trp¹⁰ | 7.97 | 4.47 | 2.98/3.08 | — | — | — | 9.76 | (H¹) |
| | | | | | | | 6.91 | (H²) |
| | | | | | | | 7.27 | (H⁴) |
| | | | | | | | 6.77 | (H⁵) |
| | | | | | | | 6.77 | (H⁶) |
| | | | | | | | 7.05 | (H⁷) |

^{a}Chemical shifts of aromatic protons were assigned using the NOESY spectrum. δ(Phe⁵H⁴) could not be assigned unambiguously due to signal overlap.

TABLE 2
³J(H^{N}H^{α}) of cyclo[21,29][D-Cys²¹,Cys²⁹]-uPA₂₁₋₃₀ in water
at 280 K. NMR-derived values and the corresponding values
calculated from the rMD trajectory are given. ³J(H^{N}H^{α})
were not employed as restraints during the rMD simulation.

| Residue | ³J(H^{N}H^{α})ₑₓₚ | ³J(H^{N}H^{α})_{calc} |
|---|---|---|
| Asn² | 9.1 | 7.1 ± 2.3 |
| Lys³ | 7.1 | 5.3 ± 2.0 |
| Tyr⁴ | 11.3 | 8.0 ± 1.9 |
| Phe⁵ | 11.9 | 9.7 ± 1.3 |
| Ser⁶ | 8.7 | 3.9 ± 3.2 |
| Asn⁷ | 9.1 | 6.5 ± 2.5 |
| Ile⁸ | 8.6 | 5.6 ± 2.4 |
| Cys⁹ | 8.7 | 9.6 ± 1.1 |
| Trp¹⁰ | 9.4 | 8.8 ± 1.7 |

TABLE 3
³J(H^{α}H^{β}) of cyclo[21,29][D-Cys²¹,Cys²⁹]-uPA₂₁₋₃₀ in water
at 280 K. NMR-derived values and the corresponding values
calculated from the rMD trajectory are given. Due to side-
chain rotation or NOESY signal overlap no diastereotopic
assignment could be made. ³J(H^{α}H^{β}) were not employed as
restraints during the rMD simulation.

| Residue | ³J(H^{α}H^{β})ₑₓₚ | | ³J(H^{α}H^{β})calc | |
|---|---|---|---|---|
| D-Cys¹ | 4.5, | 10.2 | 9.2 ± 4.3 | (proS) |
| | | | 4.6 ± 1.7 | (proR) |
| Asn² | 4.6, | 9.2 | 12.1 ± 1.6 | (proS) |
| | | | 3.1 ± 0.9 | (proR) |
| Lys³ | 6.3, | 6.4 | 7.8 ± 5.0 | (proS) |
| | | | 4.6 ± 2.4 | (proR) |
| Tyr⁴ | 6.0, | 10.3 | 3.8 ± 1.2 | (proS) |
| | | | 3.5 ± 1.2 | (proR) |
| Phe⁵ | 6.4, | 9.0 | 3.1 ± 1.7 | (proS) |
| | | | 11.8 ± 2.5 | (proR) |
| Ser⁶ | both ca. 7.0 (overlapped) | | 2.6 ± 0.7 | (proS) |
| | | | 5.1 ± 1.3 | (proR) |
| Asn⁷ | 7.4, | 7.8 | 12.0 ± 1.1 | (proS) |
| | | | 2.4 ± 0.7 | (proR) |
| Ile⁸ | 6.8 | | 6.9 ± 4.5 | |
| Cys⁹ | 5.3, | 9.5 | 8.8 ± 4.2 | (proS) |
| | | | 5.1 ± 4.6 | (proR) |
| Trp¹⁰ | 6.5, | 7.5 | 3.0 ± 1.0 | (proS) |
| | | | 6.0 ± 3.5 | (proR) |

TABLE 4
Populations of hydrogen bonds of cyclo[21,29][D-Cys²¹,Cys²⁹]-
uPA₂₁₋₃₀ in water at 280 K calculated from the rMD trajectory^{a}

| donor | acceptor | population |
|---|---|---|
| Asn²H^{N} | Ile⁸CO | 76 |
| Asn²H^{N} | Ser⁶CO | 23 |
| Lys³H^{N} | Asn²O^{δ1} | 42 |
| Tyr⁴H^{N} | Asn²O^{δ1} | 60 |
| Phe⁵H^{N} | Asn²O^{δ1} | 52 |
| Ser⁶H^{N} | Tyr⁴CO | 14 |
| Ser⁶HO^{γ} | Phe⁵CO | 10 |
| Asn⁷H^{N} | Ser⁶O^{γ} | 49 |
| Asn⁷H^{N} | Phe⁵CO | 14 |
| Ile⁸H^{N} | Phe⁵CO | 59 |
| Trp¹⁰H^{N} | Ile⁸CO | 13 |

^{a}Hydrogen bonds are defined by a distance between donor and acceptor of D_{A,D} ≦ 2.8Å and an angle between the vectors NH and HO of δ = 180° ± 60°.

TABLE 5

| | | | | | | | | |
|---|---|---|---|---|---|---|---|---|
| ATOM | 1 | N | CYS | 1 | 23.523 | 11.953 | 18.425 | N |
| ATOM | 2 | CA | CYS | 1 | 23.062 | 13.252 | 18.958 | C |
| ATOM | 3 | C | CYS | 1 | 21.585 | 13.483 | 18.552 | C |
| ATOM | 4 | O | CYS | 1 | 20.678 | 12.784 | 19.019 | O |
| ATOM | 5 | CB | CYS | 1 | 23.252 | 13.289 | 20.488 | C |
| ATOM | 6 | SG | CYS | 1 | 22.725 | 14.883 | 21.147 | S |
| ATOM | 7 | 1H | CYS | 1 | 23.021 | 11.171 | 18.860 | H |
| ATOM | 8 | 2H | CYS | 1 | 24.524 | 11.803 | 18.593 | H |
| ATOM | 9 | 3H | CYS | 1 | 23.374 | 11.885 | 17.413 | H |
| ATOM | 10 | HA | CYS | 1 | 23.713 | 14.040 | 18.528 | H |
| ATOM | 11 | 1HB | CYS | 1 | 24.309 | 13.117 | 20.767 | H |
| ATOM | 12 | 2HB | CYS | 1 | 22.664 | 12.494 | 20.985 | H |
| ATOM | 13 | N | ASN | 2 | 21.356 | 14.487 | 17.688 | N |
| ATOM | 14 | CA | ASN | 2 | 19.992 | 14.928 | 17.286 | C |
| ATOM | 15 | C | ASN | 2 | 19.459 | 14.098 | 16.077 | C |
| ATOM | 16 | O | ASN | 2 | 20.213 | 13.751 | 15.160 | O |
| ATOM | 17 | CB | ASN | 2 | 20.072 | 16.450 | 16.982 | C |
| ATOM | 18 | CG | ASN | 2 | 18.746 | 17.206 | 16.792 | C |
| ATOM | 19 | OD1 | ASN | 2 | 17.679 | 16.832 | 17.284 | O |
| ATOM | 20 | ND2 | ASN | 2 | 18.801 | 18.316 | 16.078 | N |
| ATOM | 21 | H | ASN | 2 | 22.201 | 14.959 | 17.348 | H |
| ATOM | 22 | HA | ASN | 2 | 19.316 | 14.803 | 18.158 | H |
| ATOM | 23 | 1HB | ASN | 2 | 20.612 | 16.974 | 17.794 | H |
| ATOM | 24 | 2HB | ASN | 2 | 20.709 | 16.604 | 16.093 | H |
| ATOM | 25 | 1HD2 | ASN | 2 | 17.920 | 18.827 | 15.955 | H |
| ATOM | 26 | 2HD2 | ASN | 2 | 19.714 | 18.549 | 15.668 | H |
| ATOM | 27 | N | LYS | 3 | 18.143 | 13.809 | 16.086 | N |
| ATOM | 28 | CA | LYS | 3 | 17.468 | 12.989 | 15.036 | C |
| ATOM | 29 | C | LYS | 3 | 17.537 | 13.619 | 13.608 | C |
| ATOM | 30 | O | LYS | 3 | 18.126 | 13.016 | 12.707 | O |
| ATOM | 31 | CB | LYS | 3 | 16.015 | 12.678 | 15.502 | C |
| ATOM | 32 | CG | LYS | 3 | 15.273 | 11.590 | 14.686 | C |
| ATOM | 33 | CD | LYS | 3 | 13.783 | 11.403 | 15.047 | C |
| ATOM | 34 | CE | LYS | 3 | 13.507 | 10.961 | 16.499 | C |
| ATOM | 35 | NZ | LYS | 3 | 12.077 | 10.675 | 16.708 | N |
| ATOM | 36 | H | LYS | 3 | 17.625 | 14.200 | 16.880 | H |
| ATOM | 37 | HA | LYS | 3 | 18.005 | 12.020 | 14.997 | H |
| ATOM | 38 | 1HB | LYS | 3 | 16.029 | 12.352 | 16.559 | H |
| ATOM | 39 | 2HB | LYS | 3 | 15.416 | 13.608 | 15.501 | H |
| ATOM | 40 | 1HG | LYS | 3 | 15.322 | 11.843 | 13.609 | H |
| ATOM | 41 | 2HG | LYS | 3 | 15.806 | 10.625 | 14.784 | H |
| ATOM | 42 | 1HD | LYS | 3 | 13.239 | 12.343 | 14.836 | H |
| ATOM | 43 | 2HD | LYS | 3 | 13.359 | 10.657 | 14.349 | H |
| ATOM | 44 | 1HE | LYS | 3 | 14.101 | 10.063 | 16.752 | H |
| ATOM | 45 | 2HE | LYS | 3 | 13.818 | 11.750 | 17.208 | H |
| ATOM | 46 | 1HZ | LYS | 3 | 11.769 | 9.875 | 16.145 | H |
| ATOM | 47 | 2HZ | LYS | 3 | 11.872 | 10.459 | 17.689 | H |
| ATOM | 48 | 3HZ | LYS | 3 | 11.491 | 11.474 | 16.443 | H |
| ATOM | 49 | N | TYR | 4 | 16.958 | 14.821 | 13.423 | N |
| ATOM | 50 | CA | TYR | 4 | 16.972 | 15.552 | 12.126 | C |
| ATOM | 51 | C | TYR | 4 | 18.303 | 16.239 | 11.688 | C |
| ATOM | 52 | O | TYR | 4 | 18.450 | 16.486 | 10.488 | O |
| ATOM | 53 | CB | TYR | 4 | 15.732 | 16.489 | 12.011 | C |
| ATOM | 54 | CG | TYR | 4 | 15.605 | 17.804 | 12.830 | C |
| ATOM | 55 | CD1 | TYR | 4 | 15.897 | 17.873 | 14.199 | C |
| ATOM | 56 | CD2 | TYR | 4 | 15.027 | 18.917 | 12.206 | C |
| ATOM | 57 | CE1 | TYR | 4 | 15.599 | 19.021 | 14.929 | C |
| ATOM | 58 | CE2 | TYR | 4 | 14.728 | 20.064 | 12.939 | C |
| ATOM | 59 | CZ | TYR | 4 | 15.010 | 20.111 | 14.301 | C |
| ATOM | 60 | OH | TYR | 4 | 14.677 | 21.218 | 15.035 | O |
| ATOM | 61 | H | TYR | 4 | 16.517 | 15.217 | 14.261 | H |
| ATOM | 62 | HA | TYR | 4 | 16.792 | 14.782 | 11.349 | H |
| ATOM | 63 | 1HB | TYR | 4 | 15.629 | 16.730 | 10.935 | H |
| ATOM | 64 | 2HB | TYR | 4 | 14.822 | 15.888 | 12.212 | H |
| ATOM | 65 | HD1 | TYR | 4 | 16.336 | 17.041 | 14.723 | H |
| ATOM | 66 | HD2 | TYR | 4 | 14.773 | 18.898 | 11.154 | H |
| ATOM | 67 | HE1 | TYR | 4 | 15.817 | 19.054 | 15.988 | H |
| ATOM | 68 | HE2 | TYR | 4 | 14.256 | 20.901 | 12.448 | H |
| ATOM | 69 | HH | TYR | 4 | 14.748 | 21.000 | 15.967 | H |
| ATOM | 70 | N | PHE | 5 | 19.255 | 16.535 | 12.601 | N |
| ATOM | 71 | CA | PHE | 5 | 20.570 | 17.136 | 12.237 | C |
| ATOM | 72 | C | PHE | 5 | 21.699 | 16.228 | 12.809 | C |
| ATOM | 73 | O | PHE | 5 | 21.830 | 16.066 | 14.025 | O |
| ATOM | 74 | CB | PHE | 5 | 20.683 | 18.606 | 12.731 | C |
| ATOM | 75 | CG | PHE | 5 | 19.648 | 19.636 | 12.221 | C |
| ATOM | 76 | CD1 | PHE | 5 | 19.300 | 19.710 | 10.864 | C |
| ATOM | 77 | CD2 | PHE | 5 | 19.051 | 20.526 | 13.123 | C |
| ATOM | 78 | CE1 | PHE | 5 | 18.352 | 20.629 | 10.427 | C |
| ATOM | 79 | CE2 | PHE | 5 | 18.115 | 21.456 | 12.680 | C |
| ATOM | 80 | CZ | PHE | 5 | 17.762 | 21.504 | 11.334 | C |
| ATOM | 81 | H | PHE | 5 | 19.024 | 16.289 | 13.570 | H |
| ATOM | 82 | HA | PHE | 5 | 20.681 | 17.175 | 11.134 | H |
| ATOM | 83 | 1HB | PHE | 5 | 20.685 | 18.599 | 13.838 | H |
| ATOM | 84 | 2HB | PHE | 5 | 21.683 | 18.987 | 12.451 | H |
| ATOM | 85 | HD1 | PHE | 5 | 19.753 | 19.045 | 10.142 | H |
| ATOM | 86 | HD2 | PHE | 5 | 19.314 | 20.508 | 14.172 | H |
| ATOM | 87 | HE1 | PHE | 5 | 18.077 | 20.662 | 9.382 | H |
| ATOM | 88 | HE2 | PHE | 5 | 17.656 | 22.137 | 13.381 | H |
| ATOM | 89 | HZ | PHE | 5 | 17.028 | 22.218 | 10.991 | H |
| ATOM | 90 | N | SER | 6 | 22.500 | 15.622 | 11.912 | N |
| ATOM | 91 | CA | SER | 6 | 23.473 | 14.549 | 12.270 | C |
| ATOM | 92 | C | SER | 6 | 24.681 | 14.973 | 13.162 | C |
| ATOM | 93 | O | SER | 6 | 24.844 | 14.411 | 14.248 | O |
| ATOM | 94 | CB | SER | 6 | 23.898 | 13.794 | 10.987 | C |
| ATOM | 95 | OG | SER | 6 | 24.543 | 14.644 | 10.042 | O |
| ATOM | 96 | H | SER | 6 | 22.276 | 15.833 | 10.934 | H |
| ATOM | 97 | HA | SER | 6 | 22.909 | 13.802 | 12.863 | H |
| ATOM | 98 | 1HB | SER | 6 | 24.574 | 12.955 | 11.238 | H |
| ATOM | 99 | 2HB | SER | 6 | 23.018 | 13.327 | 10.503 | H |
| ATOM | 100 | HG | SER | 6 | 23.863 | 15.240 | 9.717 | H |
| ATOM | 101 | N | ASN | 7 | 25.501 | 15.956 | 12.731 | N |
| ATOM | 102 | CA | ASN | 7 | 26.610 | 16.522 | 13.562 | C |
| ATOM | 103 | C | ASN | 7 | 26.149 | 17.380 | 14.792 | C |
| ATOM | 104 | O | ASN | 7 | 26.812 | 17.346 | 15.834 | O |
| ATOM | 105 | CB | ASN | 7 | 27.587 | 17.286 | 12.617 | C |
| ATOM | 106 | CG | ASN | 7 | 28.971 | 17.655 | 13.200 | C |
| ATOM | 107 | OD1 | ASN | 7 | 29.544 | 16.946 | 14.027 | O |
| ATOM | 108 | ND2 | ASN | 7 | 29.555 | 18.758 | 12.754 | N |
| ATOM | 109 | H | ASN | 7 | 25.235 | 16.372 | 11.831 | H |
| ATOM | 110 | HA | ASN | 7 | 27.175 | 15.659 | 13.969 | H |
| ATOM | 111 | 1HB | ASN | 7 | 27.787 | 16.669 | 11.718 | H |
| ATOM | 112 | 2HB | ASN | 7 | 27.082 | 18.193 | 12.226 | H |
| ATOM | 113 | 1HD2 | ASN | 7 | 30.478 | 18.976 | 13.145 | H |
| ATOM | 114 | 2HD2 | ASN | 7 | 29.042 | 19.302 | 12.052 | H |
| ATOM | 115 | N | ILE | 8 | 25.018 | 18.109 | 14.682 | N |
| ATOM | 116 | CA | ILE | 8 | 24.388 | 18.875 | 15.799 | C |
| ATOM | 117 | C | ILE | 8 | 23.851 | 17.907 | 16.913 | C |
| ATOM | 118 | O | ILE | 8 | 23.318 | 16.832 | 16.618 | O |
| ATOM | 119 | CB | ILE | 8 | 23.300 | 19.830 | 15.170 | C |
| ATOM | 120 | CG1 | ILE | 8 | 23.944 | 20.995 | 14.350 | C |
| ATOM | 121 | CG2 | ILE | 8 | 22.286 | 20.404 | 16.187 | C |
| ATOM | 122 | CD1 | ILE | 8 | 23.000 | 21.854 | 13.490 | C |
| ATOM | 123 | H | ILE | 8 | 24.569 | 18.049 | 13.762 | H |
| ATOM | 124 | HA | ILE | 8 | 25.170 | 19.522 | 16.245 | H |
| ATOM | 125 | HB | ILE | 8 | 22.699 | 19.224 | 14.473 | H |
| ATOM | 126 | 1HG1 | ILE | 8 | 24.511 | 21.656 | 15.032 | H |
| ATOM | 127 | 2HG1 | ILE | 8 | 24.705 | 20.583 | 13.661 | H |
| ATOM | 128 | 1HG2 | ILE | 8 | 22.792 | 21.032 | 16.942 | H |
| ATOM | 129 | 2HG2 | ILE | 8 | 21.507 | 21.016 | 15.701 | H |
| ATOM | 130 | 3HG2 | ILE | 8 | 21.738 | 19.610 | 16.729 | H |
| ATOM | 131 | 1HD1 | ILE | 8 | 22.260 | 22.403 | 14.099 | H |
| ATOM | 132 | 2HD1 | ILE | 8 | 23.572 | 22.612 | 12.924 | H |
| ATOM | 133 | 3HD1 | ILE | 8 | 22.443 | 21.249 | 12.756 | H |
| ATOM | 134 | N | CYS | 9 | 23.995 | 18.332 | 18.186 | N |
| ATOM | 135 | CA | CYS | 9 | 23.537 | 17.555 | 19.367 | C |
| ATOM | 136 | C | CYS | 9 | 22.612 | 18.431 | 20.257 | C |
| ATOM | 137 | O | CYS | 9 | 23.085 | 19.261 | 21.041 | O |
| ATOM | 138 | CB | CYS | 9 | 24.777 | 17.030 | 20.126 | C |
| ATOM | 139 | SG | CYS | 9 | 24.310 | 16.032 | 21.558 | S |
| ATOM | 140 | H | CYS | 9 | 24.448 | 19.248 | 18.285 | H |
| ATOM | 141 | HA | CYS | 9 | 22.977 | 16.653 | 19.045 | H |
| ATOM | 142 | 2HB | CYS | 9 | 25.424 | 17.860 | 20.475 | H |
| ATOM | 143 | 1HB | CYS | 9 | 25.404 | 16.402 | 19.463 | H |
| ATOM | 144 | N | TRP | 10 | 21.287 | 18.212 | 20.147 | N |
| ATOM | 145 | CA | TRP | 10 | 20.289 | 18.723 | 21.125 | C |
| ATOM | 146 | CB | TRP | 10 | 19.858 | 20.208 | 20.932 | C |
| ATOM | 147 | CG | TRP | 10 | 19.268 | 20.673 | 19.587 | C |
| ATOM | 148 | CD1 | TRP | 10 | 17.996 | 20.331 | 19.072 | C |
| ATOM | 149 | CD2 | TRP | 10 | 19.770 | 21.626 | 18.709 | C |
| ATOM | 150 | NE1 | TRP | 10 | 17.707 | 21.016 | 17.880 | N |
| ATOM | 151 | CE2 | TRP | 10 | 18.815 | 21.818 | 17.675 | C |
| ATOM | 152 | CE3 | TRP | 10 | 20.961 | 22.400 | 18.735 | C |
| ATOM | 153 | CZ2 | TRP | 10 | 19.049 | 22.773 | 16.657 | C |
| ATOM | 154 | CZ3 | TRP | 10 | 21.170 | 23.334 | 17.717 | C |
| ATOM | 155 | CH2 | TRP | 10 | 20.230 | 23.514 | 16.693 | C |
| ATOM | 156 | C | TRP | 10 | 19.102 | 17.737 | 21.221 | C |
| ATOM | 157 | OT1 | TRP | 10 | 18.533 | 17.327 | 20.183 | O |
| ATOM | 158 | OT2 | TRP | 10 | 18.722 | 17.377 | 22.358 | O |
| ATOM | 159 | HN | TRP | 10 | 21.026 | 17.528 | 19.428 | H |
| ATOM | 160 | HA | TRP | 10 | 20.763 | 18.692 | 22.126 | H |
| ATOM | 161 | 1HB | TRP | 10 | 20.732 | 20.840 | 21.169 | H |
| ATOM | 162 | 2HB | TRP | 10 | 19.134 | 20.474 | 21.727 | H |
| ATOM | 163 | HD1 | TRP | 10 | 17.297 | 19.666 | 19.558 | H |
| ATOM | 164 | HE1 | TRP | 10 | 16.867 | 20.943 | 17.297 | H |
| ATOM | 165 | HE3 | TRP | 10 | 21.702 | 22.270 | 19.511 | H |
| ATOM | 166 | HZ2 | TRP | 10 | 18.325 | 22.939 | 15.875 | H |
| ATOM | 167 | HZ3 | TRP | 10 | 22.081 | 23.915 | 17.709 | H |
| ATOM | 168 | HH2 | TRP | 10 | 20.425 | 24.240 | 15.918 | H |

List of atomic coordinates in units of 0.1 nm. Column 2 indicates atom number, column 3 atom name, column 4 residue type, column 5 residue number, column 6,7,8 the x,y,z coordinates and column 9 indicates atom type.

1 K. Wüthrich, M. Billeter, W. Braun, J. Mol. Biol. 1983, 169, 949-961.

2 Y. Kim, J. H. Prestegard, J. Magn. Reson. 1989, 84, 9-13.

3 A. T. Brünger X-PLOR Manual. Version 3.1.; Yale University Press: Cambridge, Mass., 1992.

4 M. Nilges, G. M. Clore, A. M. Gronenborn, FEBS Lett. 1988, 239, 129-136.

5 M. Nilges, J. Kuszewski, A. T. Brünger, Sampling properties of simulated annealing and distance geometry. In Computational aspects of the study of biological macromolecules by nuclear magnetic resonance spectroscopy, J. C. Hoch, F. M. Poulsen and C. Redfield (Hrsg.), Plenum Press, New York, N.Y., 1991, 451-61.

6 Torda, R. M. Scheek, W. F. van Gunsteren, Chem. Phys. Lett. 1989, 157, 289-294.

7 Torda, R. M. Scheek, W. F. van Gunsteren, J. Mol. Biol. 1990, 214, 223-230.

8 D. A. Pearlman, P. A. Kollmann, J. Mol. Biol. 1991, 220, 457-79.

9 A. P. Nanzer, W. F. van Gunsteren, A. E. Torda, J. Biomol. NMR 1995, 6, 313-320.

10 A. P. Hansen, A. M. Petros, R. P. Meadows, D. G. Nettesheim, A. P. Mazar, E. T. Olejnizak, R. X. Xu, T. M. Perderson, J. Henkin, S. W. Fesik, Biochemistry 1994, 33, 4847-4864.

11 G. Mueller, M. Gurrath, M. Kurz, H. Kessler, Proteins 1993,. 15, 235-51.

12 J. S. Richardson, Adv. Protein Chem. 1981, 34, 167-339.

13 P. N. Lewis, F. A. Momany, H. A. Scheraga, Biochem. Biophys. Acta 1973, 303, 211-29.

14 D. C. Rees, M. Lewis, W. N. Lipscomb, J. Mol. Biol. 1983, 168, 367-87.

15 F. C. Bernstein, T. F. Koetzle, G. J. Williams, E. J. Meyer, M. D. Brice, J. R. Rodgers, O. Kennard, T. Shimanouchi, M. Tasumi, J. Mol. Biol. 1977, 112, 535-42.

16 M. Levitt, J. Am. Chem. Soc. 1978, 17, 4277-85.

17 T. M. Gray, B. W. Matthews, J. Mol. Biol. 1984, 175, 75-81.

18 J. M. Thornton, B. L. Sibanda, M. S. Edwards, D. J. Barlow, Bioessays 1988, 8, 63-9.

19 C. M. Wilmot, J. M. Thornton, J. Mol. Biol. 1988, 203, 221-32.

20 E. G. Hutchinson, J. M. Thornton, Protein Sci. 1994, 3, 2207-16.

21 R. A. Laskowski, M. W. Macarthur, D. S. Moss, J. M. Thornton, Appl. Crystallogr. 1993, 26, 283-91.

22 M. Bürgle, M. Koppitz, C. Riemer, H. Kessler, B. König, U. H. Weidle, J. Kellermann, F. Lottspeich, H. Graeff, M. Schmitt, L. Goretzki, U. Reuning, O. Wilhelm, V. Magdolen. Biol. Chem. 1997, 378:231-237

23 R. J. Goodson, M. V. Doyle, S. E. Kaufman, S. Rosenberg, Proc. Natl. Acad. Sci. U.S.A. 1994, 91, 7129-33.

24 M. Ploug, S. Ostergaard, L. B. L. Hansen, A. Holm, K. Dano, Biochemistry 1998, 37, 3612-22.

FIG. 1: Histogram of NOE-derived distance restraints per residue. Intraresidue (black), short-range (gray; |i−j|<5, where i and j are residue numbers of participating residues) and long-range (white; |i−j|>5) NOEs are given.

FIG. 2: Radial distribution functions g(r) of water oxygens around backbone amide protons. A steep rise of g(r) at r=2.0 Å, as observed for Lys³, Ser⁶, and Cys⁹, indicates solvent exposition of the respective amide proton, allowing for the formation of hydrogen bonds with the solvent The gradual rise of g(r) seen in the plots for Asn², Phe⁵, and Ile⁸ results from shielding of the respective amide proton from solvent, accomplished by intramolecular hydrogen bonds or vicinity of side chains. Experimentally determined temperature dependances of the amide proton chemical shifts (Δδ/ΔT [−ppb/K], see plots) correlate well with the calculated radial distribution functions.

FIG. 3: Stereoview of cyclo[21,29][D-Cys²¹,Cys²⁹]UPA₂₁₋₃₀. Different atom types are shown in the following manner hydrogen (small white spheres), carbon (large white spheres), nitrogen (black spheres), oxygen (gray spheres). The three-dimensional structure is characterized by a hydrophobic cluster involving Tyr⁴, Phe⁵, Ile⁸, and Trp¹⁰, and two type βI turns centered at Lys³, Tyr⁴ and Ser⁶, Asn⁷, respectively.

FIG. 4: χ¹ angles in the course of the two 200 ps rMD simulations starting from different initial velocities. Each plot is split by a vertical line, displaying the data of simulation 1 and simulation 2 on the left-hand and the right-hand side, respectively.

FIG. 5: Ramachandran plots generated from the two 200 ps rMD simulations starting from different initial velocities.

FIG. 6: Comparison of the NMR solution structures of the ATF of uPA and cyclo[21,29][D-Cys²¹,Cys²⁹]uPA₂₁₋₃₀. C^{α}-C^{β} vectors of Tyr⁴, Phe⁵ and Ile⁸ of the peptide were superimposed on the corresponding protein residues (RMSD of C^{α},C^{β} atoms after superposition: 0.6 Å).

Please amend claims 4 and 6-9 as follows:

1. (Original) Use of the 3D-structure of cyclo[21,29][D-Cys21Cys29]-uPA₂₁₋₃₀ for the design of uPA antagonists.

2. (Original) uPA antagonists derived from the drug lead cyclo[21,29][D-Cys21Cys29]-uPA₂₁₋₃₀, comprising at least part of the 3D-structure of the drug lead and comprising at least one non-peptidic structural unit with respect to either peptide bonds or amino acid side chains.

3. (Original) uPA antagonists according to claim 2, wherein conformation stabilizing cycles are introduced into the peptide, such that Ramachandran angles actually found in the drug lead are stabilized.

4. (Currently amended) uPA antagonists according to claim 2 or 3, wherein β-turn mimetics replace the tetrapeptides Asn-Lys-Tyr-Phe and/or Phe-Ser-Asn-Ile.

5. (Original) uPA antagonists according to claim 4, wherein the β-D-glucose or the cyclohexane scaffold are used as β-turn mimetics.

6. (Currently amended) uPA antagonists according to any one of claims 2 to 5, wherein Lys3/Tyr4 and/or Ser6/Asn7 are replaced with α-helix inducing dipeptide mimetics.

7. (Currently amended) uPA antagonists according to any one of claims 2 to 6, wherein the molecule or a part of the molecule is a carbapeptide.

8. (Currently amended) uPA antagonists according to any one of claims 2 to 7, wherein the molecule or a part of the molecule is an azapeptide.

9. (Currently amended) uPA antagonists according to any one of claims 2 to 8, wherein the molecule or a part of the molecule is a peptoid.

10. (Original) uPA antagonists according to claim 2, wherein the conformation of the drug lead is stabilized by additional bridges between amino acids or their analogues that are not adjacent in the peptide sequence.
